(19) United States
(12) Patent Application Publication
(10) Pub. No.: US 2024/0230543 A9
(43) Pub. Date: Jul. 11, 2024

(54) COLOR-CHANGING SENSOR FOR GAS DETECTION AND METHOD FOR MANUFACTURING SAME

(71) Applicant: INDUSTRY-UNIVERSITY COOPERATION FOUNDATION HANYANG UNIVERSITY ERICA CAMPUS, Ansan-si (KR)

(72) Inventors: Yong-Ho CHOA, Seongnam-si (KR); Byungkwon JANG, Pyeongtaek-si (KR); Gwang-Myeong GO, Ansan-si (KR); Hong-Baek CHO, Ansan-si (KR); Bongyoung YOO, Seongnam-si (KR)

(73) Assignee: INDUSTRY-UNIVERSITY COOPERATION FOUNDATION HANYANG UNIVERSITY ERICA CAMPUS, Ansan-si (KR)

(21) Appl. No.: 18/514,456

(22) Filed: Nov. 20, 2023

Related U.S. Application Data
(63) Continuation of application No. PCT/KR2022/007209, filed on May 20, 2022.

(30) Foreign Application Priority Data
May 20, 2021 (KR) 10-2021-0064866
Apr. 27, 2022 (KR) 10-2022-0051805
Apr. 27, 2022 (KR) 10-2022-0051806
Apr. 27, 2022 (KR) 10-2022-0051807

(51) Int. Cl. G01N 21/78; G01N 33/00

(52) U.S. Cl. CPC G01N 21/78; G01N 33/0027

(57) ABSTRACT

The present invention relates to a color-changing sensor for gas detection and a method for manufacturing same and, more particularly, to a color-changing sensor that can be used for detecting hydrogen gas, in which when the sensor comes into contact with hydrogen gas, color conversion occurs so that it is possible to easily check the presence of hydrogen gas, and when the sensor is not in contact with hydrogen gas by removal of hydrogen gas or the like, the sensor may exhibit a reversible characteristic of being restored to an original color and an irreversible characteristic of not being restored to an original color.

### CROSS-REFERENCE TO RELATED APPLICATIONS

This application claims the priority benefit of Korean Patent Application No. 10-2021-0064866 filed on May 20, 2021, Korean Patent Application No. 10-2022-0051805 filed on Apr. 27, 2022, Korean Patent Application No. 10-2022-0051806 filed on Apr. 27, 2022 and Korean Patent Application No. 10-2022-0051807 filed on Apr. 27, 2022 in the Korean Intellectual Property Office, the disclosures of which are incorporated herein by reference.

This application is a PCT Continuation By-Pass application of PCT Application No. PCT/KR2022/007209 filed on May 20, 2022, in the Korean Intellectual Property Office, the entire disclosures of which are incorporated herein by reference for all purposes.

### BACKGROUND

### 1. Field

The present disclosure relates to a color-changing sensor for gas detection and a method for manufacturing the same, and more specifically, to a color-changing sensor, which is a sensor for detecting hydrogen gas and changes color when detecting hydrogen gas, and a method for manufacturing the same.

### 2. Description of the Related Art

Recently, there have been extensive researches to use hydrogen gas as a renewable clean energy source as an alternative to petroleum. Hydrogen fuel, which has high combustion heat and low ignition energy and burns completely, is considered a candidate for a future energy source.

However, since hydrogen is highly volatile and has flammable and explosive properties, when concentration of hydrogen exceeds a critical threshold, it is dangerous. Meanwhile, since hydrogen is a colorless, odorless, tasteless, flammable gas, it cannot be detected by human senses. When the concentration of hydrogen in the atmosphere exceeds 4%, it surpasses the explosion limit, so when there are an ignition source and oxygen, it causes risks of explosion and fire.

Therefore, a hydrogen sensor is essential for the safe use of hydrogen as a fuel.

Various types of hydrogen sensors have been reported, among which electrical sensors are the most widely used. Specifically, electrical sensors using palladium with high hydrogen adsorption capacity are commonly used. However, palladium, when being exposed to hydrogen of low concentration, has the alpha (a) phase and changes electrical conductivity proportionally with the concentration of hydrogen in proportion to the concentration of hydrogen. However, palladium, when being exposed to hydrogen of high concentration, is changed from the alpha (a) phase to the beta (p) phase. Since palladium in the beta (p) phase is not changed in electrical conductivity in proportion to the hydrogen concentration, the palladium cannot be used as a hydrogen detecting material.

Furthermore, when palladium is changed from the alpha (a) phase to the beta (p) phase, it undergoes volume expansion. Accordingly, when palladium is repeatedly exposed to high concentrations of hydrogen, it leads to cracks and breakage in the hydrogen detection layer, so the palladium cannot detect hydrogen. Electrical sensors using palladium as a hydrogen detection material can detect only hydrogen with concentration of less than about 4%.

Recently, various forms of optical detection methods have been researched and developed, such as films and substrates that change color upon exposure to hydrogen gas. However, to quantify slight hydrogen detection levels, additional components, such as optical sensors which are optically transparent or capable of detecting color changes, are required.

In addition, there is additional problem in that it is not easy to detect hydrogen due to the irreversible characteristic where the color does not return to its original state when getting in contact with hydrogen after color changes due to contact with hydrogen and due to the low visibility that there is no distinct color difference before and after the color change.

### SUMMARY

It is an object of the present invention to provide a color-changing sensor for gas detection and a method for manufacturing the same.

It is another object of the present invention to provide a color-changing sensor for gas detection, which is a color-changing sensor capable of being used to detect hydrogen gas, can easily verify the presence of hydrogen gas through color-change when contacting with hydrogen gas, and show reversible characteristics to return to its original color or irreversible characteristics to maintain the changed color in case of non-contact with hydrogen gas due to the removal of hydrogen gas.

It is a further object of the present invention to provide a color-changing sensor for gas detection, which is applicable to various fields since being used in various types, such as a tape type or a spray type, is convenient to use as detecting hydrogen in the air without the need for electrical power, and can be used not only at room temperature but also at sub-zero temperature.

To accomplish the above-mentioned objects, according to the present invention, there is provided a color-changing sensor for gas detection including: an adhesive layer; a catalytic layer; and a thermochromic layer, wherein the catalytic layer includes a porous support bonded to metal particles.

The metal particles are selected from a group consisting of precious metal catalysts, precious metal alloy catalysts, non-precious metal catalysts, non-precious metal alloy catalysts, metal oxides, metal chlorides, metal complexes, and mixtures thereof.

The porous support is selected from the group consisting of graphene, carbon allotropes, ceramic oxides, and mixtures thereof.

The thermochromic layer includes thermochromic particles, and the thermochromic particles include dye and developer.

The thermochromic layer includes a thermochromic coating composition, and the thermochromic coating composition comprises thermochromic particles, a binder, and a solvent.

The adhesive layer has a porous structure.

The color-changing sensor further includes a spacer.

The gas is selected from the group consisting of hydrogen gas, methane gas, ethane gas, propane gas, butane gas, and mixtures thereof.

The catalytic layer generates heat at temperature between 30 and 110° C. when the concentration of hydrogen in the air is between 1 and 4%.

The color-changing sensor detects exposure of the target gas under conditions ranging from −20 to 20° C.

In another aspect of the present invention, there is provided a color-changing tape for gas detection including a color-changing sensor.

In another aspect of the present invention, there is provided a method for manufacturing a color-changing sensor for gas detection including the steps of: manufacturing a porous support, on the surface of which metal particles are bonded; bonding the porous support to one side of an adhesive layer; and preparing a thermochromic coating composition by dissolving thermochromic particles and a binder in a solvent and coating thermochromic coating composition on one side of the porous support, on the surface of which metal particles are bonded, to form a thermochromic layer.

The metal particles are selected from a group consisting of precious metal catalysts, precious metal alloy catalysts, non-precious metal catalysts, non-precious metal alloy catalysts, metal oxides, metal chlorides, metal complexes, and mixtures thereof.

The porous support is selected from the group consisting of graphene, carbon allotropes, ceramic oxides, and mixtures thereof.

The thermochromic particles include dye and developer.

The adhesive layer has a porous structure.

To accomplish the above-mentioned objects, according to the present invention, there is provided a color-changing sensor for gas detection including: an adhesive layer; and a porous thermochromic catalytic layer, wherein the porous thermochromic catalytic layer includes a composite structure, and the composite structure comprises a porous support to which metal particles are bonded.

In another aspect of the present invention, there is provided a method for manufacturing a color-changing sensor for gas detection, including the steps of: manufacturing a composite structure with metal particles bonded on the surface of a porous support; preparing a thermochromic coating solution by mixing the composite structure, thermochromic particles, a binder, and a solvent; coating the thermochromic coating solution on PET to form a porous thermochromic catalytic layer; and bonding the porous thermochromic catalytic layer to one side of an adhesive layer.

In another aspect of the present invention, there is provided a color-changing sensor for gas detection including: an adhesive layer; a web-type catalytic layer; and a thermochromic layer, wherein the web-type catalytic layer includes a nanofiber support, and metal catalysts are bonded on the surface of the nanofiber support.

In another aspect of the present invention, there is provided a method for manufacturing a color-changing sensor for gas detection, including the steps of: manufacturing a nanofiber support by spinning a nanofiber support solution; coating metal catalyst on the nanofiber support to form a web-type catalytic layer; bonding the web-type catalytic layer to one side of an adhesive layer; and preparing a thermochromic coating composition by dissolving thermochromic particles and a binder in a solvent and coating the thermochromic coating composition on one side of the web-type catalytic layer including metal catalyst to form a thermochromic layer.

According to the present disclosure, the color-changing sensor for gas detection, which is a color-changing sensor capable of being used to detect hydrogen gas, can easily verify the presence of hydrogen gas through color-change when contacting with hydrogen gas, and show reversible characteristics to return to its original color in case of non-contact with hydrogen gas due to the removal of hydrogen gas or irreversible characteristics.

Furthermore, the color-changing sensor is applicable to various fields since being used in various types, such as a tape type or a spray type, is convenient to use as detecting hydrogen in the air without the need for electrical power, and can be used not only at room temperature but also at sub-zero temperature.

### BRIEF DESCRIPTION OF THE DRAWINGS

FIG. 1 is a view of a color-changing sensor for gas detection according to an embodiment of the present invention.

FIGS. 2A and 2B show a view of a composite structure 110 included in a catalyst layer 100 of the color-changing sensor for gas detection according to an embodiment of the present invention.

FIGS. 3A and 3B show a view of a detection mechanism of target gas in the color-changing sensor for gas detection according to an embodiment of the present invention.

FIG. 4 is a graph showing a change rate of heat generation to gas concentration of the catalyst layer 100 according to an embodiment of the present invention.

FIG. 5 is a view of a color-changing sensor for gas detection which further includes a diffusion space layer type composite structure (layer-by-layer type & combined layer type with spacers).

FIG. 6 is a view illustrating a manufacturing method of a color-changing sensor for gas detection according to an embodiment of the present invention.

FIGS. 7A and 7B show a view illustrating a porous support and nano-sized metal particles bonded to the surface of the porous support, according to an embodiment of the present invention.

FIG. 8 is a schematic diagram illustrating a structure of a color-changing sensor including a web-type catalyst layer.

FIGS. 9A, 9B, 9C, 9D and 9E show a view illustrating various structures in the web-type catalyst layer.

FIG. 10 is a view of a color-changing sensor for gas detection according to another embodiment of the present invention.

FIG. 11 is a view illustrating a color-changing sensor for gas detection of a combined layer type, which further includes a diffusion space layer type composite structure (layer-by-layer type & combined layer type with spacers).

FIG. 12 is a view illustrating a manufacturing method of a color-changing sensor for gas detection according to another embodiment of the present invention.

FIGS. 13A, 13B and 13C show color change performance evaluation results of an irreversible color-changing sensor which includes a heat color-changing layer by different target temperatures.

FIGS. 14A, 14B and 14C show color change performance evaluation results of a reversible color-changing sensor according to an embodiment of the present invention.

FIG. 15 shows target gas detection performance evaluation results at a tube joint part of the color-changing sensor according to an embodiment of the present invention.

FIG. 16 shows performance evaluation results of the color-changing sensor according to an embodiment of the present invention.

FIG. 17 shows performance evaluation results of the color-changing sensor according to an embodiment of the present invention.

FIG. 18 shows performance evaluation results of the color-changing sensor according to an embodiment of the present invention.

FIG. 19 shows performance evaluation results of the color-changing sensor according to an embodiment of the present invention.

### DETAILED DESCRIPTION

Hereinafter, embodiments of the present invention will be described in detail below so that a person of ordinary skill in the art can easily implement. However, the present disclosure may be implemented in various ways without being limited to the embodiments.

Advantages and features of the present disclosure and methods accomplishing the advantages and features will become apparent from the following detailed description of exemplary embodiments with reference to the accompanying drawings. However, the present invention is not limited to exemplary embodiment disclosed herein but will be implemented in various forms. The exemplary embodiments are provided so that the present invention is completely disclosed, and a person of ordinary skilled in the art can fully understand the scope of the present invention. Therefore, the present invention will be defined only by the scope of the appended claims.

In the present invention, the concentration of hydrogen in the air is referred to in terms of volume concentration (vol %).

Hydrogen has been widely used across various industries, including fuel cell vehicles, the steel industry, and the semiconductor industry, is gaining attention as a promising energy source due to lack of greenhouse gas emissions, and is rapidly increasing in the amount used.

However, due to hydrogen's inherent colorless, odorless, and tasteless properties, and explosive nature, when hydrogen gas escapes into the air, it can cause equipment damage or human accidents.

Additionally, the escape of hydrogen may cause deformation or destruction at joint portions of transport pipes.

As research and development in hydrogen vehicles are activated, there is growing interest in hydrogen gas storage, management, leak prevention, and detection.

Therefore, the use of hydrogen gas requires more precise and complete management and handling. Accurate and rapid detection of hydrogen gas in concentrations below 4%, which is outside the explosive range, is essential to prevent accidents caused by the hydrogen leak.

In general, the hydrogen leak in the air is detected by hydrogen sensors or hydrogen detection sensors in conventional technology. The conventional hydrogen sensors and detection sensors have high manufacturing costs due to the structure of a substrate and are difficult to rapidly detect exposed hydrogen.

In addition, since silicon substrates and silicon-on-insulator (SOI) substrates are used, there is a limitation in using with flexibility, so the utility of the products is deteriorated. To overcome the problems, there is a need for hydrogen sensors that do not rely on substrates and electricity and offer greater freedom and flexibility in use.

A hydrogen sensor using platinum-based oxides like palladium (Pd) can detect hydrogen through the color change of the platinum-based oxides. However, the hydrogen sensor has a problem in that in that it is not easy to detect hydrogen due to the irreversible characteristics where the color does not return to its original state when getting in contact with hydrogen after color changes due to contact with hydrogen and due to the low visibility that there is no distinct color difference before and after the color change.

The present invention relates to a color-changing sensor for gas detection that is flexible, does not utilize a substrate, and has color-changing properties with high visibility. The color-changing sensor for gas detection, when gas is detected, makes it easy to confirm gas detection, and can be repeatedly used several times due to the variability of returning to its original color after removal of target gas.

Hereinafter, for convenience of description, a color-changing sensor for gas detection according to an embodiment is referred to as a layer-by-layer type color-changing sensor, and a color-changing sensor for gas detection according to another embodiment is referred to as a combined layer type color-changing sensor.

In the following description, it should be understood that components that perform the same functions or operations are given the same reference numerals, and the functions, operations, manufacturing methods, and experimental results of components having the same reference numerals are the same unless specifically described otherwise.

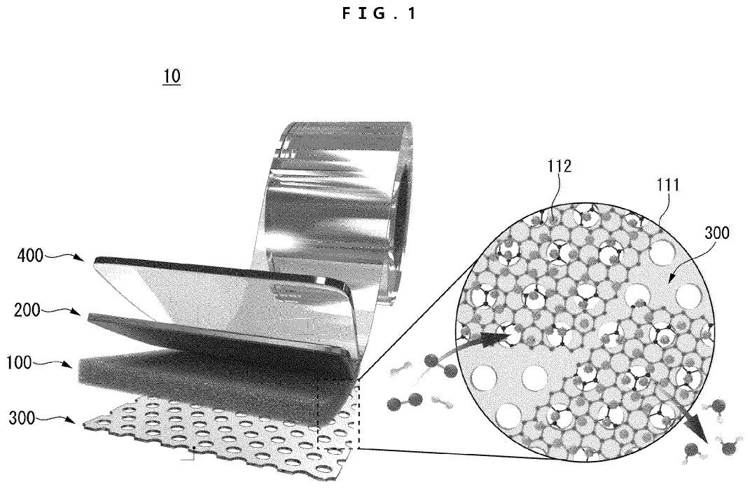

Hereinafter, referring to the attached drawings, the color-changing sensor for gas detection according to an embodiment will be described. FIG. 1 is a view of a color-changing sensor for gas detection according to an embodiment of the present invention.

FIG. 1 illustrates the structure of a layer-by-layer type color-changing sensor. The layer-by-layer type color-changing sensor 10 includes a catalyst layer (porous hydrogen exothermic reaction layer) 100 in which a porous support and metal particles are bonded, and a thermochromic layer 200. The catalyst layer 100 and the thermochromic layer 200 are stacked and interconnected to be adjacent to each other.

The color-changing sensor for gas detection according to an embodiment can include a porous adhesive layer 300. The adhesive layer 300 has a porous structure, in which air containing target gas can freely move to the catalyst layer 100 of the color-changing sensor.

The adhesive layer 300 can be made of any material with adhesive properties, including epoxy resin, but is not limited thereto.

The catalyst layer 100 includes a porous composite structure. The composite structure can include a porous support bonded with metal particles. When the target gas is in contact with the metal particles bonded with the porous support, an exothermic reaction occurs, and heat generated by the exothermic reaction in the catalyst layer 100 is transferred to the thermochromic layer 200 through the porous support.

The thermochromic layer 200 changes color depending on the temperature. As described above, the thermal energy is transferred to the thermochromic layer 200 by the exothermic reaction of the catalyst layer 100.

When temperature of the thermochromic layer 200 exceeds a predetermined target temperature due to thermal energy, the color of the thermochromic layer 200 changes. In this instance, the target temperature refers to the temperature at which the color of the thermochromic layer 200 changes, and the target temperature varies depending on the composition and ratio of the materials forming the thermochromic layer 200.

The thermochromic layer 200 may exhibit reversible characteristics that changes color when the temperature exceeds a preset level and returns to its original color when the temperature decreases, or irreversible characteristics that does not return to the original (previous) color once changing the color when the temperature exceeds target temperature.

The layers forming the color-changing sensor for gas detection according to an embodiment of the invention will be described in detail as follows:

Protective Layer

Referring to FIG. 1, the color-changing sensor for gas detection according to an embodiment may further include a protective layer 400. The protective layer 400 is provided on one side of the thermochromic layer 200 and can prevent damage and wear to the color-changing sensor.

The protective layer 400 can be made of transparent or translucent materials to facilitate observation of the color change of the thermochromic layer 200.

The protective layer 400 can be made of translucent materials such as polyethylene terephthalate (PET) or polyethylene (PE).

Adhesive Layer

The adhesive layer 300 included in the color-changing sensor for gas detection is intended to adhere the color-changing sensor for gas detection to a gas storage part, a gas transport part, or the like, especially a joint part of gas transport pipes, a hydrogen gas storage part and a hydrogen gas transport part in a hydrogen vehicle.

A conventional color-changing sensor for gas detection, which is stacked on a substrate, is deteriorated in flexibility. That is, a silicon substrate and a silicon-on-insulator (SOI) substrate which have been used generally have a limitation in bending, so it is difficult to attach them closely to joints and other such parts.

Therefore, the color-changing sensor for gas detection according to the present invention does not use a substrate but uses the adhesive layer 300, and allows for free use in any shape or location where gas detection is required without limitation in forms of used portions.

The adhesive layer 300 has a porous structure that allows the target gas to diffuse to the catalyst layer 100.

The adhesive layer 300 has a porous structure, and may be made of any adhesive material, for example, epoxy resin, but is not limited thereto, and can be made of all materials which can be formed in a porous structure and has adhesive force capable of being adhered on various materials.

The adhesive layer 300 according to an embodiment can include a porous material and an adhesive material. For example, the adhesive layer 300 can be formed by applying epoxy resin, which has adhesive properties, to the porous material, such as carbon fabric or a flexible lattice-structured substrate. In this case, any material having the same function as the porous material and the adhesive substance can be used without limitation.

In another embodiment, the adhesive layer 300 can form the porous structure by producing an adhesive material, such as epoxy, into a film form and forming a fine hole in the produced film.

In this instance, the fine hole can be formed by a physical method or a chemical method, and can be formed by any method capable of forming a hole in the adhesive material. For instance, an adhesive layer 300 with holes facilitating the gaseous diffusion can be manufactured by forming holes in an adhesive film made of epoxy using a punch uniformly dotted from micro to millimeter scales.

Catalyst Layer

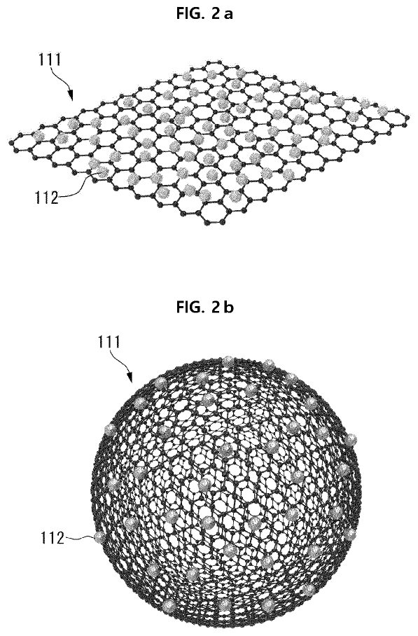

FIGS. 2A and 2B show a view of a composite structure included in the catalyst layer of the color-changing sensor for gas detection according to an embodiment of the present invention.

Referring to FIGS. 1, 2A and 2B, the catalyst layer 100 can include a composite structure 111 as described above.

The composite structure 110 may include a porous support 111 and nano-sized metal particles 112 bonded with the surface of the porous support 111.

Depending on components, the composite structure 110 can have a plate type structure as illustrated in FIG. 2A or a spherical structure as illustrated in FIG. 2B, but is not limited thereto.

The porous support 111 facilitates the diffusion of gas components due to the wide specific surface area and porosity, and provides a space in which the metal particles 112 can be deposited uniformly. Additionally, the porous support 111 is bonded with the metal particles 112 deposited on the surface of the porous support 111 through physical bonding, ionic bonding, hydrogen bonding, or covalent bonding.

The porous support 111 can be selected from a group consisting of graphene, carbon allotropes, ceramic oxides, and mixtures thereof, and more specifically, can be carbon allotropes, such as graphene, graphite, carbon nanotube, carbon black, ketjen black, and activated carbon, and porous ceramic oxides, such as alumina, silica, ceria, and the like. Preferably, the porous support 111 can be selected from any materials capable of easily transferring heat generated by the exothermic reaction of metal particles 112 with the target gas to the thermochromic layer 200 without being limited by graphene or the exemplary materials.

The structure of the porous support 111 can vary depending on components. For example, if the porous support 111 is graphene, the porous support 111 may have a plate type structure as illustrated in FIG. 2A, and if it is a carbon allotrope or porous ceramic, the porous support 111 may have a spherical structure as illustrated in FIG. 2B.

The metal particles 112 can be selected from a group consisting of precious metal catalysts, precious metal alloy catalysts, non-precious metal catalysts, non-precious metal alloy catalysts, metal oxides, metal chlorides, metal complexes, and mixtures thereof.

The metal particles 112 may include precious metal catalysts, such as Pt, Pd, Rh, Ru, Ir, and Os, precious metal alloy catalysts, such as PtCo, PtFe, PtRu, AuPt, PtCu, PtNb, PtNi, PdCu, PdIr, and IrRh, non-precious metal catalysts, such as Ni, W, Zn, Ag, Ti, Co, Mo, Al, Fe, V, Sb, Sn, Bi, Mn, Cu, and Ba, non-precious metal alloy catalysts, such as NiFe, NiCu, NiCo, MoNi4, and WNi4, and oxides, chlorides, or complexes of the above metals, but are not limited to the above examples, and can include any capable of undergoing an exothermic reaction with the target gas without being limited by the above.

The size of the metal particles 112 may range from 0.1 to 900 nm. Using metal particles 112 within the range can increase the contact area with the target gas, facilitate bonding with the porous support 111, and enhance the efficiency of heat transfer.

The metal particles 112 bonded with the porous support 111 can represent 0.1 to 50 wt % based on the total weight of the catalyst layer 100. When the metal particles 112 are used within the range, heat is generated by the exothermic reaction with the target gas, and the generated heat can be transferred through the porous support 111 to the thermochromic layer 200.

The bonding between the porous support 111 and the metal particles 112 can be physical bonding, ionic bonding, hydrogen bonding, or covalent bonding, but is not limited thereto, and any bonding method by which the metal particles 112 bonded by external force are not easily removed and can generate heat by the exothermic reaction without hindering contact with the target gas can be used without limitation.

The catalyst layer 100 generates heat at temperature between 30 and 110° C. when the concentration of hydrogen in the air is between 1 and 4%, preferably between 39 and 102° C. Within the range, when the catalyst layer 100 reacts with hydrogen to generate heat, the heat is transferred to the thermochromic layer 200, and the transferred heat causes the color of the thermochromic layer 200 to change.

Thermochromic Layer

The thermochromic layer 200 changes color depending on temperature. The thermochromic layer 200 includes thermochromic particles that change color at the target temperature.

Hereinafter, referring to the drawings, the color-changing mechanism of the thermochromic layer 200 and the gas detection mechanism of the color-changing sensor for gas detection 10 according to an embodiment will be described.

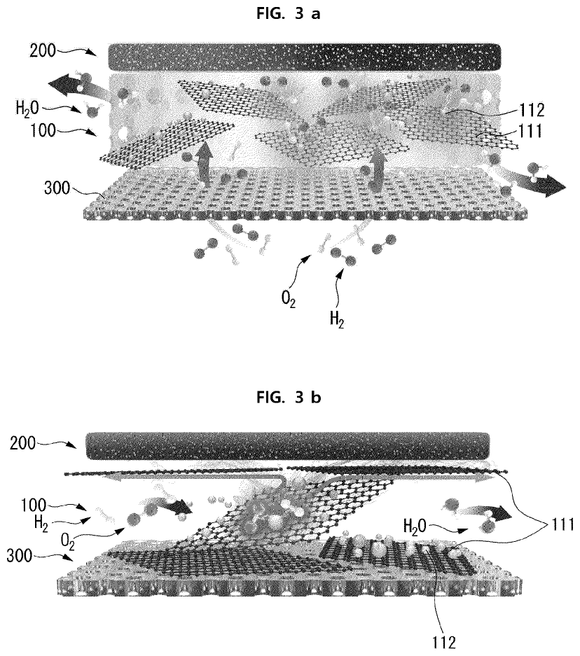

FIGS. 3A and 3B show a view illustrating a gas detection mechanism of the target gas applied to the color-changing sensor for gas detection according to an embodiment of the invention. In this instance, the target gas is specifically hydrogen gas.

As illustrated in FIG. 3A, hydrogen gas molecules exposed in the air pass through the porous adhesive layer 300 via diffusion and reach the composite structure 110. The hydrogen gas reaching the composite structure 110 arrives at the surface of metal particles 112 uniformly dispersed on the surface of the porous support 111. The hydrogen gas reaching the surface of the metal particles 112 is chemically adsorbed through dissociative adsorption on the catalyst surface. The dissociatively adsorbed hydrogen gas undergoes an exothermic reaction to produce water molecules through a surface reaction with oxygen adsorbed in the air.

The heat generated by the exothermic reaction on the surface of the metal particles 112, as illustrated in FIG. 3B, is transmitted through the porous support 111 to the thermochromic layer 200.

The porous support 111 which includes wide pores on the surface thereof provides a wide deposition area where the nano-sized metal particles 112 can bond, and provides a diffusion space for hydrogen and oxygen molecules, thereby activating the reaction. Furthermore, the porous support 111 is uniformly dispersed within the catalyst layer 100, and effectively and uniformly transfers the reaction heat to the thermochromic layer.

Thermochromic Particles

The thermochromic layer 200 includes thermochromic particles that change color at the target temperature.

The color change of the thermochromic layer 200 according to changes of temperature can exhibit either reversible or irreversible characteristics depending on components.

Here, the reversible characteristics mean that color of is changed when the temperature of the thermochromic particles exceeds the target temperature and is returned to the original color when the temperature drops below the target temperature. The irreversible characteristics mean that color of is changed when the temperature of the thermochromic particles exceeds the target temperature and is not returned to the original (previous) color once the color is changed.

In this instance, the target temperature can vary depending on the components of the thermochromic layer 200.

The thermochromic layer 200 can include thermochromic particles with reversible characteristics, or thermochromic particles with irreversible characteristics. Alternatively, the thermochromic layer 200 can include both of thermochromic particles with reversible characteristics and thermochromic particles with irreversible characteristics.

Specifically, the thermochromic layer 200 with reversible characteristics may include thermochromic particles containing dye and developer, and the thermochromic layer 200 with irreversible characteristics may include thermochromic particles additionally containing waxes.

The dye is specifically a leuco dye, and the leuco dye reacts with compounds which donate hydrogen ions or hydroxide ions, thereby causing a structural change in the dye molecules to lead to color change.

Specifically, the leuco dye can be selected from a group consisting of phenolphthalein, methylene blue, methyl orange, methyl red, phenol red, bromothymol blue, bromophenol blue, and mixtures thereof, but any dye that changes structure and leads to color change by reacting with a compound which donate hydrogen ions or hydroxide ions can be used without limitation.

The compound which changes the structure of dye molecules is referred to as developer. The degree of reaction of the developer changes according to temperature. Therefore, the degree of reaction is low at lower temperature, the developer shows the original color of the dye, but when temperature increases, the reaction proceeds to lead to a color change. Accordingly, the range of color change is adjusted by the amount of developer.

The developer can use all of compounds including a phenol functional group, such as phenol, 4-t-butylphenol, 4-t-octylphenol, 2-ethylphenol, 3-ethylphenol, 4-ethylphenol, o-cresol, m-cresol, p-cresol, 2,5-xylenol, 3,4-xylenol, 3,5-xylenol, 2,3,5-trimethylphenol, 3-methyl-6-t-butylphenol, 2-naphthol, 1,3-dihydroxynaphthalene, bisphenol-A, and mixtures thereof, preferably, 2-naphthol, but is not limited to thereto.

The reversible thermochromic particles can be prepared in the form of a microemulsion by mixing dye, developer, and solvent.

In this instance, the solvent can be polyethylene glycol, but is not limited thereto, and any material capable of dissolving the dye and the developer can be used without limitation.

For example, the reversible thermochromic particles may be composed of dye, developer, and polyethylene glycol in a weight ratio of 1:2:150 to 1:10:150, but the weight ratio may vary depending on the target temperature, and adjustment of the target temperature will be further described below.

The irreversible thermochromic particles can be prepared in the form of a microemulsion by mixing dye, developer, wax, and solvent.

The wax is a material for fixing a structural change of dye molecules, and when the temperature rises and the developer changes the structure of the dye molecules, fixes the structural change of the dye molecules.

The wax can be one among cetyl alcohol, eicosanol, and C30-50 alcohol, but is not limited thereto, and any substance capable of fixing the structural change of the dye molecules can be used as wax.

When the temperature of the thermochromic particles rises above the melting point, the wax melts and the developer inside the wax flows out and reacts with the dye. Since such a reaction which is an irreversible reaction maintains the changed color of the dye, the irreversible reaction makes the thermochromic particles operate irreversibly.

That is, the wax inhibits the reaction between the dye and the developer, and once the temperature exceeds the melting point, the wax can no longer inhibit the reaction and generates thermochromism. Thereafter, when the temperature drops below the melting point, the reacted state of the dye and the developer is fixed as it is, so even if the temperature drops below the target temperature again, the color change is fixed.

Therefore, since the color is changed only when the temperature of the wax rises and the wax melts, the target temperature can vary according to the selection of wax.

For example, since the melting point of cetyl alcohol is 49.3° C., if cetyl alcohol is used as wax, the target temperature becomes above 49.3° C. If eicosanol with the melting point of 64 to 66° C., the target temperature becomes above 64 to 66° C., and if C30-50 alcohol with the melting point of 85 to 90° C., the target temperature becomes above 85 to 90° C. (Experimental results related to the above can be found through the following Tables 2 and 3.)

For instance, irreversible thermochromic particles may be composed of dye, developer, wax, and polyethylene glycol in a weight ratio of 1:2:10:150 to 1:10:10:150, but the weight ratio can vary depending on the color change temperature.

Thermochromic Particles: Target Temperature Selectivity

The thermochromic layer 200 can have target temperature selectivity that changes color, so can have concentration selection of the target gas. That is, the thermochromic particles can vary the target temperature can vary depending on weight and materials.

In an embodiment, thermochromic particles included in the thermochromic layer 200 have a target temperature in the range of 40 to 90° C. Thermochromic particles with a target temperature of 40° C. do not change color below 40° C. but can change color above 40° C.

Specifically, thermochromic particles of the present invention can exhibit target temperatures of 50° C., 70° C., or 90° C. Here, the target gas can be hydrogen gas.

As described above, hydrogen gas causes an exothermic reaction when reacting with the metal particles 112 in the catalyst layer 100. When the heat generated through the reaction is transferred to the thermochromic layer 200, temperature of the thermochromic layer 200 rises. When the temperature of the thermochromic particles rises above the target temperature, the color of the thermochromic layer 200 changes.

The exothermic reaction that raises the temperature of the thermochromic layer 200 is influenced by the concentration of the target gas. The higher the concentration of the target gas, the more vigorous the exothermic reaction in the catalyst layer 100, thereby raising the temperature of the thermochromic layer 200. Conversely, as the target gas concentration decreases, the exothermic reaction in the catalyst layer 100 diminishes, and the temperature of the thermochromic layer 200 also relatively decreases.

Therefore, by adjusting the target temperature, concentration selectivity of the target gas can be achieved.

The heat generation temperatures of the catalyst layer 100 according to the hydrogen concentration in an embodiment of the invention are as illustrated in Table 1.

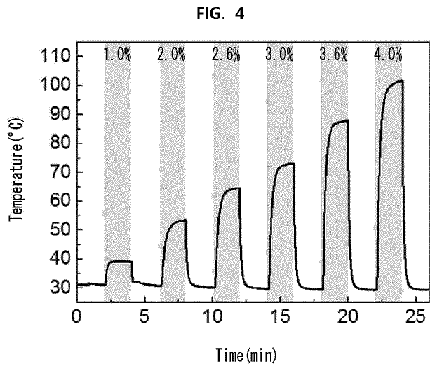

FIG. 4 is a graph showing the rate of heat generation change in the catalyst layer 100 according to the gas concentration according to an embodiment of the invention.

The heating performance of the catalyst layer 100 was measured using an IR temperature detector. An IR thermometer was installed on the top of a chamber containing the catalyst layer 100, and then, the heating temperature of the catalyst layer was monitored in real-time by flowing hydrogen gas at specified hydrogen concentration into the chamber.

As a result of the measurement, as illustrated in FIG. 4, when hydrogen gas with concentration of 1 to 4% were introduced into the catalyst layer 100, it was confirmed that, as gas concentration increased, the exothermic characteristics of the catalyst layer 100 also increased. Specifically, the numerical increase in the heating temperature according to the hydrogen concentration is shown in Table 1.

TABLE 1

| Hydrogen | |
|---|---|
| concentration(vol %) | Temperature(° C.) |
| 1.0 | 39.1 |
| 2.0 | 52.3 |
| 2.6 | 64.3 |
| 3.0 | 72.8 |
| 3.6 | 87.4 |
| 4.0 | 101.1 |

In the present invention, the heat generation temperature of the catalyst layer 100 gradually increased from 39° C. depending on the concentration of hydrogen in the air, and when the concentration of hydrogen in the air was 4%, the heat generation temperature of the catalyst layer 100 increased up to 101.1° C. Therefore, in the experimental example of the present invention, it was confirmed that the exothermic reaction of the catalyst layer 100 linearly increased with high sensitivity according to the hydrogen concentration.

As described above, since the temperature of the thermochromic layer 200 varies with the concentration of the target gas, the concentration of the detected gas can be selected by adjusting the target temperature.

More specifically, when the exposure level of hydrogen gas is at 1% concentration in the air, the heat generation temperature of the catalyst layer 100 is 39.1° C., at 2% concentration, the heating temperature is 52.8° C., and at 4.0% concentration, the heating temperature is 101.1° C.

Therefore, when thermochromic particles are configured to have a target temperature of 39.1° C. or lower, hydrogen gas at concentration below 1% can be detected. When the target temperature is set to 52.8° C., hydrogen gas at concentration of 2% or more can be detected. Additionally, thermochromic particles is configured to have a target temperature of 101.1° C., hydrogen gas at concentration of 4% or more can be detected.

The higher the target temperature is set, the higher the concentration of the target gas needs to be for detection, and the lower the target temperature is set, the lower the concentration of the target gas can be for detection.

Therefore, according to an embodiment of the invention, the target temperature can be adjusted according to the usage environment and the safe gas management concentration of the color-changing sensor for gas detection 10, thereby enhancing the usability of the color-changing sensor 10.

In case of reversible thermochromic particles, the target temperature can be adjusted using the weight range of dye, developer, and polyethylene glycol. Similarly, in case of irreversible thermochromic particles, the target temperature can be adjusted using the weight range of dye, developer, wax, and polyethylene glycol. Additionally, the irreversible thermochromic particles can adjust the target temperature by changing the type of wax.

The content of the components for manufacturing the thermochromic particles can be as shown in Table 2. Here, examples 1 to 3 are for reversible thermochromic particles, and examples 4 to 6 are for irreversible thermochromic particles.

TABLE 2

| | Leuco | | Polyethylene | Wax | Wax |
|---|---|---|---|---|---|
| | dye | Developer | glycol | type | weight |
| Example 1 | 1 | 10 | 150 | — | — |
| Example 2 | 1 | 5 | 150 | — | — |
| Example 3 | 1 | 2 | 150 | — | — |
| Example 4 | 1 | 5 | 150 | cetyl | 10 |
| | | | | alcohol | |
| Example 5 | 1 | 5 | 150 | Eicosanol | 10 |
| Example 6 | 1 | 5 | 150 | C30-50 | 10 |
| | | | | alcohol | |

The color change temperature of the color-changing sensor manufactured according to Examples 1 to 6 is as illustrated in Table 3.

TABLE 3

| | Exam- | Exam- | Exam- | Exam- | Exam- | Exam- |
|---|---|---|---|---|---|---|
| | ple 1 | ple 2 | ple 3 | ple 4 | ple 5 | ple 6 |
| color change | 50 | 70 | 90 | 50 | 70 | 90 |
| temperature | | | | | | |
| (° C.) | | | | | | |

As described above, the developer is a substance that changes the structure of dye molecules, changes the degree of reaction according to temperature, and varies the reaction temperature according to the weight ratio.

That is, as in examples 1 to 3, it is confirmed that the target temperature of the thermochromic particles increases as the ratio of the developer to the leuco dye decreases.

Preferably, for detection of hydrogen gas, the developer of the thermochromic particles according to an embodiment of the invention can have twice to ten times the content of the leuco dye.

Additionally, the thermochromic layer 200 of the present invention can be used without being significantly affected by environments using color-changing sensors. That is, the thermochromic layer 200 can be used not only at room temperature but also at sub-zero temperatures.

Specifically, the thermochromic layer 200 can detect the exposure of the target gas at temperatures ranging from −20 to 20° C. and can change the color. It means that the performance of the thermochromic layer does not deteriorate even at sub-zero temperatures, and the color-changing characteristic can be maintained due to the heat transferred from the heating layer.

When utilizing the above feature, the color-changing sensor can be highly useful even under sub-zero conditions where the target gas is stored without performance degradation.

Spacer Layer

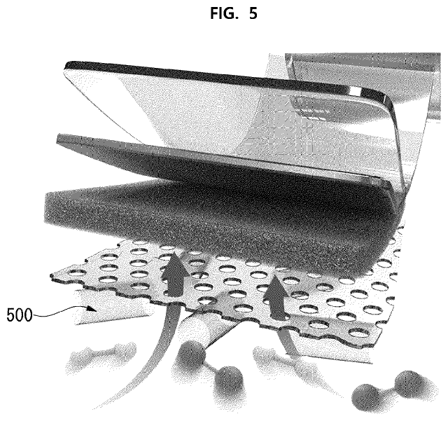

FIG. 5 is a view of a color-changing sensor for gas detection which further includes a diffusion space layer type composite structure (layer-by-layer type & combined layer type with spacers).

Referring to FIG. 5, the color-changing sensor for gas detection 10 according to an embodiment, which has the layer-by-layer type structure, may additionally include a spacer 500.

Specifically, the spacer layer 500 can be added beneath the adhesive layer 300 to ensure diffusion space for hydrogen, and increase a space to introduce oxygen from the air. Accordingly, a large amount of the target gas is secured and introduced into the catalyst layer 100 to increase the amount of the target gas introduced into the catalyst layer 100, thereby increasing the thermal sensitivity.

The spacer layer 500 is arranged on one side of the adhesive layer 300 (specifically, the opposite side of the side adhered to the catalyst layer 100). The spacer layer 500 can be formed from a material that can create a specific space between the adhesive layer 300 and the part to which the adhesive layer 300 is attached. For example, the spacer layer 500 can be manufactured by applying silica particles on one side of the adhesive layer 300, but the manufacturing method is not limited thereto.

Manufacturing of Color-Changing Sensor for Gas Detection 10

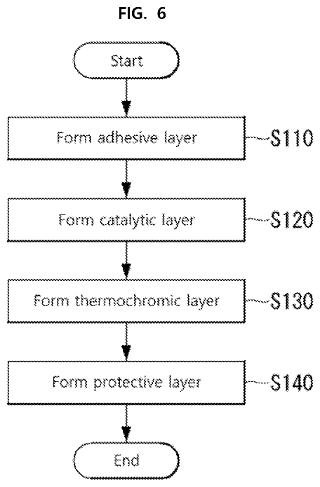

Hereinafter, a manufacturing method of the color-changing sensor for gas detection according to an embodiment will be described in detail. FIG. 6 is a view illustrating the manufacturing method of the color-changing sensor for gas detection according to an embodiment of the present invention.

Referring to FIG. 6, a porous adhesive layer 300 is formed (S110). The porous adhesive layer 300 according to an embodiment can be formed by applying epoxy resin, which has adhesive properties, to a porous material such as carbon fabric or a lattice structure substrate, but the porous material and the adhesive material are not limited thereto.

In another embodiment, the adhesive layer 300 can be formed to have a porous structure by manufacturing a material with adhesive strength, such as epoxy, in the form of a film, and forming micro-holes in the manufactured film.

A catalyst layer 100 is formed by stacking a composite structure 110 on the adhesive layer 300 (S120).

In an embodiment, a porous support is immersed in an ionic liquid and oxidized, and a metal precursor solution is added to the solution containing the oxidized porous support, and is refluxed. Continuously, a reducing agent is injected into the refluxed solution to reduce the metal precursor, thereby manufacturing a composite structure 110 containing a porous support with metal particles bonded on the surface thereof.

In the same way, the composite structure 110 with metal particles bonded on the surface of the porous support can be stacked on one side of the porous structured adhesive layer to form the catalyst layer.

Meanwhile, for convenience of description, it is described that the step of manufacturing the composite structure 110 is included in the step of forming the catalyst layer 100, but it should be understood that the manufacturing of the composite structure 110 can be performed in a separate manufacturing step.

The thermochromic particles are coated on the catalyst layer 100 to form the thermochromic layer 200 (S130). The thermochromic particles manufactured by the above method, a binder, and a solvent are mixed to prepare a thermochromic coating solution, and the prepared coating solution is coated on one side of the catalyst layer 100 to form the thermochromic layer 200.

Here, the binder can be polyvinyl acetate (PVA), and the solvent can be isopropyl alcohol (IPA).

In an embodiment, 0.1 g of thermochromic particles, 1 g of PVA binder, and 5 g of IPA can be mixed to prepare a thermochromic coating solution, and the prepared coating solution can be coated onto PET by a bar coating method to form the thermochromic layer 200.

It should be understood that the components and the content of the thermochromic particles used in the coating solution preparation can vary as described above, depending on the target temperature, and the reversibility or irreversibility of the color change. Additionally, for convenience of description, the step of forming the thermochromic layer 200 includes the step of preparing the thermochromic coating solution, but it should be understood that the preparation of the thermochromic coating solution can be performed in a separate manufacturing step.

A protective layer 400 is formed on one side of the thermochromic layer 200 (140). In an embodiment, the protective layer 400 can be formed by applying a translucent material such as polyethylene terephthalate (PET) or polyethylene (PE) on one side of the thermochromic layer 200.

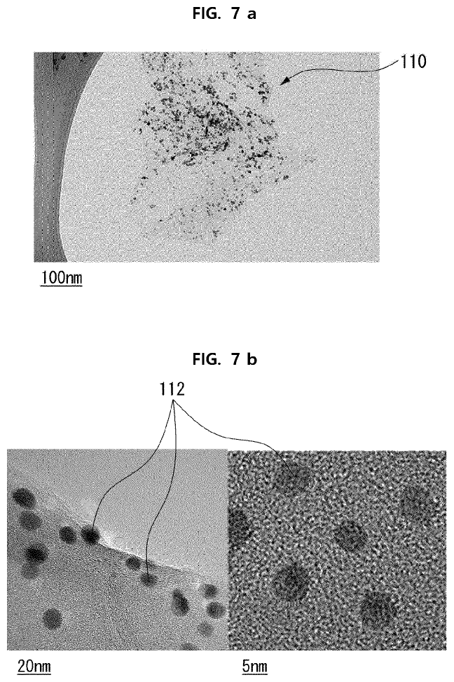

Meanwhile, although not illustrated in FIGS. 7A and 7B, an additional step of forming a spacer layer 500 on the other side of the adhesive layer 300 can be included.

Hereinafter, each manufacturing process will be described in more detail.

Manufacture of Composite Structure

Graphene and an ionic liquid were mixed and passed through a high-pressure disperser to oxidize graphene. Based on the oxidized graphene solution, 1:1 weight % of chloroplatinic acid hexahydrate solution, which was the metal precursor, was added, and the mixture was refluxed at temperature above room temperature at 830 rpm for 10 minutes. Continuously, a solution mixed with 32 mg of the reducing agent NaBH4 and 20 ml of distilled water was injected into the refluxed solution at a preset rate to reduce the metal precursor. The reduced solution was centrifuged at 1000 rpm for 5 minutes to produce the composite structure 110.

FIGS. 7A and 7B show a view illustrating a porous support and nano-sized metal particles bonded to the surface of the porous support, according to an embodiment of the present invention.

It was confirmed whether the metal particles 112 were bonded to the porous support 111 in the manufacturing embodiment of the composite structure 110. It was analyzed by using a transmission electron microscope (TEM), and the results are as illustrated in FIGS. 7A and 7B.

Specifically, as illustrated in FIG. 7A, the graphene, which is the porous support 111, and the metal particles 112 were bonded together to form the composite structure 110, and the metal particles 112 uniformly dispersed and bonded on the surface of the graphene.

As illustrated in FIG. 7B, it was confirmed that, in the composite structure 110, an interplanar spacing corresponding to the cubic structure face of platinum, which is the metal particles 112, was 0.22 nm. Consequently, it was confirmed that the composite structure 110 contained platinum particles having a single-crystal structure. Additionally, it was confirmed that the selectively grown platinum nanoparticles had a size of 5 to 10 nm.

Manufacture of Thermochromic Particles

Reversible thermochromic particles, which show reversibility in temperature, were prepared by dissolving phenolphthalein, which is the leuco dye, 2-naphthol, which is developer, and polyethylene glycol in distilled water, and heating them at 60 degrees for 30 minutes to produce a clear solution. The prepared particles were then manufactured in a powder form using a spray dryer.

In this instance, the weight ratio of the leuco dye, the developer, and polyethylene glycol can vary depending on the target temperature, and can have the weight ratio of examples 1 to 3 described above, according to the target temperature.

A clear solution which show irreversibility with temperature was prepared by dissolving phenolphthalein, 2-naphthol, which is developer, wax, and polyethylene glycol in distilled water, and heating the solution at 60 degrees for 30 minutes. The prepared particles were then manufactured in powder form using a spray dryer.

In this instance, the weight ratio of the leuco dye, the developer, polyethylene glycol, and wax can vary depending on the target temperature, and can have the weight ratio of examples 4 to 6 described above, depending on the target temperature.

Web-Type Catalyst Layer

It is described that the catalyst layer 100 of the color-changing sensor for gas detection 10 according to an embodiment includes the composite structure 110, but the structure of the catalyst layer 100 is not limited thereto. Hereinafter, referring to the attached drawings, another embodiment of the catalyst layer 100 as a web-type (Web type) catalyst layer will be described in detail.

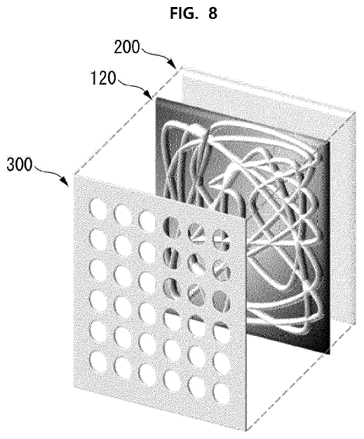

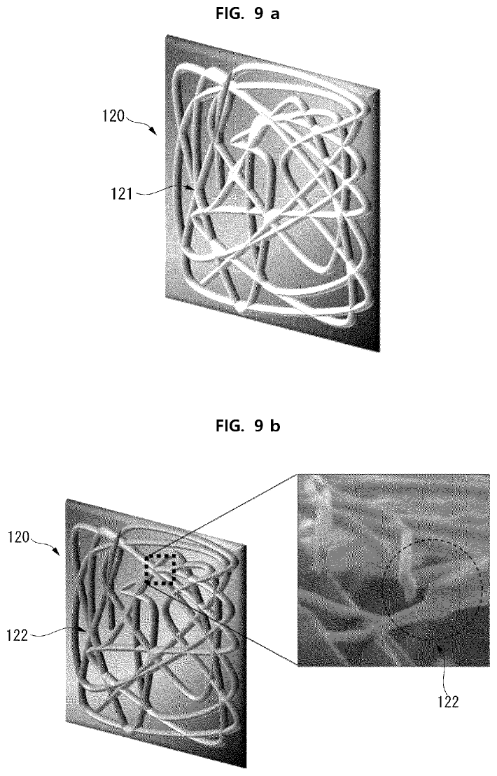

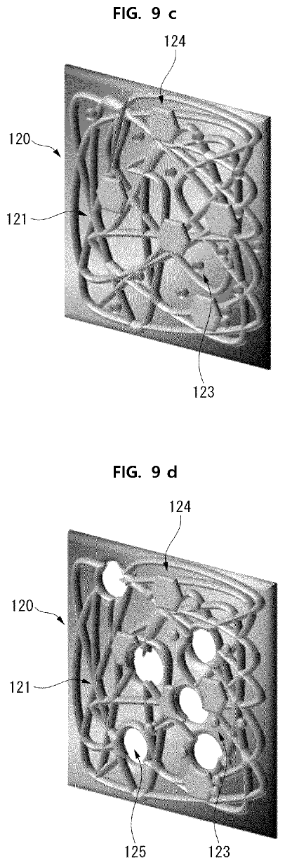

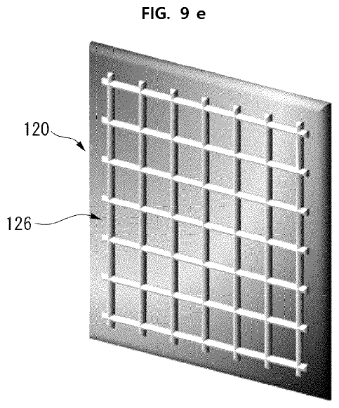

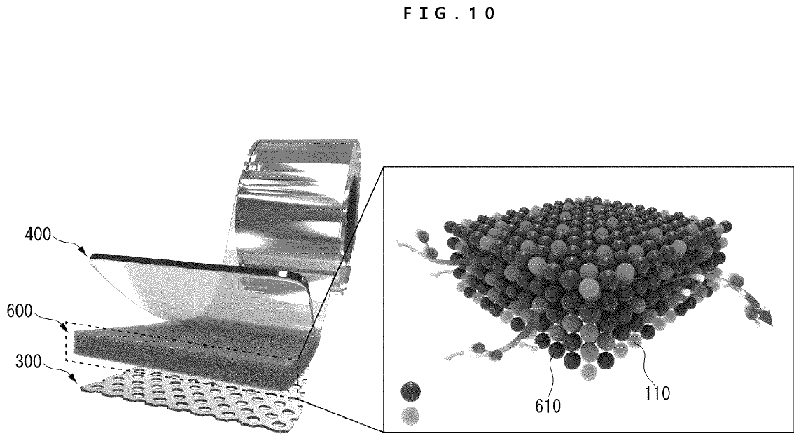

FIG. 8 is a schematic diagram illustrating a structure of a color-changing sensor including a web-type catalyst layer. FIGS. 9A to 9E show various structures of the web-type catalyst layer.

Referring to FIG. 8, the color-changing sensor for gas detection 10 includes an adhesive layer 300, a web-type catalyst layer 120, and a thermochromic layer 200. In this case, the web-type catalyst layer 120 can be formed in a complex web structure, different from the catalyst layer 100. The web-type catalyst layer 120 includes a nanofiber support 121, and the metal particles 112 can be bonded to the surface of the nanofiber support 121.

The web-type catalyst layer 120 can be further subdivided into whether the nanofiber support 121 is included, whether metal particles 123 and metal plates 124 are further included, and whether micro-holes 125 are further included. Hereinafter, referring to the drawings, various embodiments of the web-type catalyst layer will be described in more detail.

First Structure of Web-Type Catalyst Layer

As illustrated in FIG. 9A, the web-type catalyst layer 120 according to an embodiment can include a nanofiber support 121 and metal particles 112.

The web-type catalyst layer 120 according to an embodiment can have the metal particles 112 (not shown) bonded to the surface of the nanofiber support 121. The metal particles bonded to the nanofiber support 121 causes an exothermic reaction by getting in contact with the target gas, and transfers heat generated by the exothermic reaction to the thermochromic layer 200.

The metal particles 112 (not shown) can be bonded to the surface of the nanofiber support 121.

The nanofiber support 121 can be manufactured by electrospinning a polymer solution.

Specifically, the polymer solution is prepared by dissolving polyvinylpyrrolidone (PVP) in ethanol and distilled water. The nanofiber support 121 can be produced by electrospinning the polymer solution onto the adhesive layer 300.

The manufactured nanofiber support 121 can be coated with the metal particles 112 using a sputtering method to produce the web-type catalyst layer 120.

The metal particles 112 (not shown) bonded to the surface of the nanofiber support 121 generate heat by the exothermic reaction by getting contact with the target gas, and the generated heat is transferred to the thermochromic layer 200.

The metal particles 112 can be selected from a group consisting of precious metal catalysts, precious metal alloy catalysts, non-precious metal catalysts, non-precious metal alloy catalysts, metal oxides, metal chlorides, metal complexes, and mixtures thereof. The detailed description of the metal particles 112 will be omitted as being the same as previously described.

Second Structure of Web-Type Catalyst Layer

In another embodiment, the web-type catalyst layer 120, as illustrated in FIG. 9B, may include a hollow structure 122 from which the nanofiber support 121 is removed such that the interior of the catalyst layer is empty.

The hollow 122 is a space created by forming and then removing the nanofiber support 121.

Specifically, the hollow 122 is created, in the state in which the metal particles 112 (not shown) are bonded onto the surface of the nanofiber support 121, by removing only the nanofiber support 121 through heat treatment.

The web-type catalyst layer 120, from which the nanofiber support 121 is removed, retains the hollow 122 which maintains the shape of the former nanofiber support 121. The catalyst layer 100 from which the nanofiber support 121 is removed is in an empty state internally.

Third Structure of Web-Type Catalyst Layer

In yet another embodiment, as illustrated in FIG. 9C, the web-type catalyst layer 120 from which the nanofiber support 121 is removed may additionally include metal particles 123 and metal plate 124 catalysts.

The metal particles 123 and metal plate 124 catalysts are formed by applying a metal precursor solution to the web-type catalyst layer 120 from which the nanofiber support 121 is removed, and reducing the metal precursors such that metal particles 123 and metal plate 124 catalysts are additionally bonded to the surface of the catalyst layer 100.

As described above, when the metal particles 123 and metal plate 124 catalysts are additionally included, a contact area between the metal particles included in the initial web-type catalyst layer 120 and the target gas is increased, thereby leading to a faster exothermic reaction and a higher level of sensitivity.

The metal particles 123 and the metal plate 124 are additionally bonded to the web-type catalyst layer 120 of the catalyst layer 100.

The metal particles 123 and metal plate 124 can be selected from a group consisting of precious metal catalysts, precious metal alloy catalysts, non-precious metal catalysts, non-precious metal alloy catalysts, metal oxides, metal chlorides, metal complexes, and mixtures thereof.

The metal particles 123 and the metal plates 124 can be composed of precious metal catalysts such as Pt, Pd, Rh, Ru, Ir, Os; precious metal alloy catalysts such as PtCo, PtFe, PtRu, AuPt, PtCu, PtNb, PtNi, PdCu, PdIr, IrRh; non-precious metal catalysts such as Ni, W, Zn, Ag, Ti, Co, Mo, Al, Fe, V, Sb, Sn, Bi, Mn, Cu, Ba; non-precious metal alloy catalysts such as NiFe, NiCu, NiCo, MoNi4, WNi4; and oxides, chlorides, or complexes of the metals, but are not limited thereto, can include any capable of undergoing an exothermic reaction with the target gas.

The metal particles 123 and metal plates 124 are formed by applying a metal precursor solution to the web-type catalyst layer 120 and reducing the metal precursors.

The metal particles 123 and the metal plates 124 can be classified according to the size of the particles formed by the reduction of the metal precursors in the solution. That is, the metal particles 123 are bonded in the form of particles, and the metal plates 124 are bonded in the form of plates.

The web-type catalyst layer 120 further includes metal catalysts reduced in the catalyst layer 100. The metal catalysts are additionally bonded in the form of the metal particles 123 and the metal plates 124 on the surface of the catalyst layer 100, thereby increasing the contact area with the target gas.

As described above, when the contact area with the target gas is increased, a rapid exothermic reaction and a higher level of sensitivity can be shown.

Fourth Structure of Web-Type Catalyst Layer

In another embodiment, as illustrated in FIG. 9D, the web-type catalyst layer 120 which additionally includes the metal particles 123 and the metal plates 124 can further include micro-holes 125.

The micro-holes 125, which are holes connected to the surface of the hollow structure 122, increases the surface area of the catalyst layer 100, thereby increasing the contact area with the target gas. The formation of micro-holes 125 enables rapid exothermic reactions and a higher level of sensitivity.

The micro-holes 125 can be fine holes formed in the web-type catalyst layer 120, and can use a physical method or a chemical method, and any method capable of forming micro-holes 125 in the web-type catalyst layer 120 can be used without limitation.

The formation of micro-holes 125 increases the contact area with the target gas, thereby enabling rapid exothermic reactions and a higher level of sensitivity.

Fifth Structure of Web-Type Catalyst Layer

In a further embodiment, the web-type catalyst layer 120 can include nanofiber supports 121 and metal particles 112 arranged in a lattice pattern as illustrated in FIG. 9E. In this instance, the nanofiber supports 126 can be arranged in a grid pattern at preset intervals as illustrated in FIG. 9D. The metal particles (not shown) are bonded to the surface of the nanofiber supports 126 arranged in a grid pattern.

The nanofiber supports 126 can be manufactured by electrospinning a polymer solution. Specifically, the polymer solution is prepared by dissolving polyvinylpyrrolidone (PVP) in ethanol and deionized water.

The nanofiber supports 126 can be manufactured by electrospinning the polymer solution onto the adhesive layer 300 at preset intervals.

In addition, it should be understood that the grid-patterned nanofiber supports 126 may further include the hollow structure 122 from which the nanofiber support 126 is removed, the metal particles 123, the metal plates 124, and the micro-holes 125, as described in FIGS. 9b to 9d.

The method of manufacturing a color-changing sensor for gas detection including the web-type catalyst layer 120 is the same as described in FIGS. 7A and 7B, except for the step of manufacturing the catalyst layer (S120), so only the method of manufacturing the web-type catalyst layer will be described in detail.

Manufacture of Web-Type Catalyst Layer

The method of manufacturing the web-type catalyst layer 120 includes the steps of: electrospinning a nanofiber support solution to manufacture nanofiber supports; coating the nanofiber supports with metal catalysts to manufacture the web-type catalyst; and bonding the web-type catalyst layer to one surface of the adhesive layer.

Additionally, the method of manufacturing the web-type catalyst layer may further include the step of removing the nanofiber supports to create a hollow structure.

Furthermore, the method of manufacturing the web-type catalyst layer may further include the step of forming metal particles and metal plate catalysts.

Moreover, the method of manufacturing the web-type catalyst layer may further include the step of forming micro-holes.

### Manufacturing Example 1 of Web-Type Catalyst Layer

5 g of polyvinylpyrrolidone (PVP) with a molecular weight of 1,300,000 g/mol, 2 g of ethanol, and 3 g of deionized (DI) water were stirred at room temperature (25° C.) for 3 hours to prepare 10 wt % of a polymer solution. The nanofiber supports 121 were formed by electrospinning the polymer solution.

The electrospinning method was carried out using a plastic syringe of 10 ml, a tip of 30 gauge, and a voltage of 20 kV, and the polymer solution was supplied at a rate of 0.4 ml/h through the plastic syringe. Additionally, the electrospinning was performed in an environment of 40° C. temperature and 20% relative humidity.

Metal nanoparticles 112 were coated on the deposited nanofiber supports 121 using the sputtering method to manufacture a metal nanofiber catalyst. Accordingly, as illustrated in FIG. 9A, a web-type catalyst layer 120, which includes polymer nanofiber supports 121 coated with metal particles 112 reacting with hydrogen gas, can be manufactured.

### Manufacturing Example 2 of Web-Type Catalyst Layer

From the web-type catalyst layer 120 manufactured in the manufacturing example 1, the nanofiber supports 121 was removed through heat treatment at 400° C., thereby creating the hollow structure 122.

As depicted in FIG. 9B, since the metal particles 112 remain in the hollow 122 structure, the web-type catalyst layer 120 reacting with hydrogen gas was manufactured.

### Manufacturing Example 3 of Web-Type Catalyst Layer

A metal precursor solution was prepared by mixing distilled water and one of the metal particles, PtCl4 powder, at a weight percentage of 10:1.

1 mL of the metal precursor solution was applied to the web-type catalyst layer 120 from which the nanofiber supports 121 manufactured in the example 2 was removed, web-type catalyst layer 120 was dried at 120° C. and reduced at 400° C.

Through the reduction process, a multi-dimensional metal web-type catalyst layer 120 reacting with hydrogen gas, from which the nanofiber support 121 including the metal particles 123 and the metal plates 124 was removed, was manufactured as illustrated in FIG. 9C.

### Manufacturing Example 4 of Web-Type Catalyst Layer

Micro-holes 125 were formed in the multi-dimensional metal web-type catalyst layer 120 manufactured in the manufacturing example 3, as illustrated in FIG. 9D. The micro-holes 125 facilitate the gas phase diffusion of the target gas.

The micro-holes 125 were formed using a punch uniformly perforated from micro to millimeter scales. It should be understood that various physical or chemical methods can substitute the manufacturing method for micro-holes 125. Manufacturing Example 5 of Web-Type Catalyst Layer

5 g of polyvinylpyrrolidone (PVP) with a molecular weight of 1,300,000 g/mol, 2 g of ethanol, and 3 g of deionized (DI) water were stirred at room temperature (25° C.) for 3 hours to prepare 10 wt % of a polymer solution. The polymer solution was applied at preset intervals to form nanofiber supports 126 of a grid structure, as illustrated in FIG. 9E.

### Different Embodiment—Combined Layer Type

Hereinafter, with reference to the drawings, a color-changing sensor for gas detection 10 according to another embodiment will be described in detail.

FIG. 10 is a view of the structure of a combined layer type color-changing sensor for gas detection 20 according to another embodiment of the invention. The combined layer type color-changing sensor for gas detection 20 includes a porous thermochromic hydrogen exothermic reaction layer 600 in which a catalyst layer 100 and a thermochromic layer 200 are formed in a single layer.

The porous thermochromic catalyst layer 600 has porous characteristics and causes the exothermic reaction with the target gas by metal particles 112, and can change color by heat generated by the exothermic reaction.

The layer-by-layer type color-changing sensor 10 is divided into the catalyst layer 100 and the thermochromic layer 200, and the heat generated in the catalyst layer 100 moves to the thermochromic layer 200 to detect the presence of the target gas through color change. The combined layer type color-changing sensor includes the porous thermochromic catalyst layer 600 in which the catalyst layer 100 and the thermochromic layer 200 are formed in a single layer.

The porous thermochromic catalyst layer 600, as illustrated in FIG. 10, may include a composite structure 110 and thermochromic particles 610. In this instance, unless specifically stated otherwise, it is understood that the composite structure 110 and the thermochromic particles 610 are the same as described in a previous example.

The adhesive layer 300 has a porous structure, and any material with adhesive strength, such as epoxy resin, can be used, but is not limited thereto.

The gas detection mechanism of the combined layer type color-changing sensor for gas detection 20 is the same as the layer-by-layer type color-changing sensor for gas detection 20 described in FIGS. 3A and 3B. Specifically, in the combined layer structure of the present invention, the thermochromic catalyst layer 600 includes the composite structure 110 and thermochromic particles.

Therefore, as illustrated in FIG. 3A, hydrogen gas molecules exposed to the air reaches the composite structure 110 of the thermochromic catalyst layer 600 through the adhesive layer 300 by diffusion. More specifically, hydrogen gas reaches the surface of metal particles 112 uniformly dispersed on the porous support 111 of the composite structure 110. Hydrogen gas reaching the surface of the metal particles 112 adsorbed chemically through dissociative adsorption on the surface of the metal particles 112. The dissociatively adsorbed hydrogen gas undergoes an exothermic reaction with oxygen adsorbed from the air to create water molecules.

The porous support 111 includes wide pores in the surface thereof to offer a wide adsorption area for nano-sized metal particles 112 and facilitate the diffusion of hydrogen and oxygen molecules, thereby activating the reaction. Additionally, the porous support 111 is uniformly distributed within the thermochromic catalyst layer 600 to effectively and evenly transfer the reaction heat to the thermochromic particles.

The heat generated by the exothermic reaction on the surface of the metal particles 112 of the composite structure 110 is transferred to the thermochromic particles through the porous support 111, thereby causing the thermochromic particles to change color.

The color-changing sensor for gas detection according to an embodiment of the present invention may further include a protective layer 400. The protective layer 400 is provided on one surface of the thermochromic catalyst layer 600 (specifically, the opposite surface to the one where the adhesive layer 300 is provided) to prevent damage and wear of the color-changing sensor.

The protective layer 400 can be made of a transparent or translucent material to facilitate observation of color changes in the thermochromic catalyst layer 600.

Unless otherwise specified, it should be understood that the adhesive layer 300 and the protective layer 400 of the color-changing sensor for gas detection 20 according to another embodiment are the same as the color-changing sensor for gas detection 10 of the above embodiments.

Hereinafter, the thermochromic catalyst layer 600 of the color-changing sensor for gas detection 20 according to another embodiment of the invention will be described in detail.

The thermochromic catalyst layer 600 includes the composite structure 110 and thermochromic particles. In this instance, the composite structure 110 and the thermochromic particles 610 may be mixed as illustrated in FIG. 10.

As illustrated in FIGS. 2A and 2B, the composite structure 110 may include a porous support 111 and nano-sized metal particles 112 bonded to the surface of the porous support 111.

Depending on components, the composite structure 110 may have a plate-type structure as illustrated in FIG. 2A or a spherical structure as illustrated in FIG. 2B, but is not limited thereto. The porous support 111 facilitates the diffusion of gas components due to the wide specific surface area and porosity, and provides a space for uniform adsorption of the metal particles 112. Furthermore, the porous support 111 is bonded to the metal particles 112 adsorbed on the surface through physical bonding, ionic bonding, hydrogen bonding, and covalent bonding.

The porous support 111 can be selected from a group consisting of graphene, carbon allotropes, ceramic oxides, and mixtures thereof. More specifically, the porous support 111 can be graphene, graphite, carbon allotropes such as carbon nanotubes, carbon black, ketjen black, activated carbon, or the like, and porous ceramic oxides such as alumina, silica, ceria. Preferably, the porous support 111 is not limited to graphene or the above examples, but any material that allows the heat generated from the exothermic reaction of the metal particles 112 with the target gas to be easily transferred to the thermochromic layer 200 can be used without limitation.

The structure of the porous support 111 can vary depending on components thereof. For instance, if the porous support 111 is graphene, the porous support 111 has a plate-type structure as illustrated in FIG. 2A, whereas if the porous support 111 is a carbon allotrope or porous ceramic, the porous support 111 has a spherical structure as illustrated in FIG. 2B.

The metal particles 112 can be selected from a group consisting of precious metal catalysts, precious metal alloy catalysts, non-precious metal catalysts, non-precious metal alloy catalysts, metal oxides, metal chlorides, metal complexes, and mixtures thereof. The metal particles 112 can be composed of precious metal catalysts such as Pt, Pd, Rh, Ru, Ir, Os; precious metal alloy catalysts such as PtCo, PtFe, PtRu, AuPt, PtCu, PtNb, PtNi, PdCu, PdIr, IrRh; non-precious metal catalysts such as Ni, W, Zn, Ag, Ti, Co, Mo, Al, Fe, V, Sb, Sn, Bi, Mn, Cu, Ba; non-precious metal alloy catalysts such as NiFe, NiCu, NiCo, MoNi4, WNi4; and oxides, chlorides, or complexes of the metals, but are not limited thereto, and can include any capable of undergoing an exothermic reaction with the target gas.

The size of the metal particles 112 can range from 0.1 to 900 nm. The use of metal particles 112 within the range, can increase the contact area with the target gas, facilitate bonding with the porous support 111, and enhance the efficiency of heat transfer.

The metal particles 112 bonded to the porous support 111 can have 0.1 to 50 wt % based on the total weight of the composite structure 110. When the metal particles 112 is used within the range, the heat generated by the exothermic reaction with the target gas can be transmitted through the porous support 111 to the thermochromic particles.

The bonding between the porous support 111 and the metal particles 112 can be achieved by physical bonding, ion bonding, hydrogen bonding, or covalent bonding, but is not limited thereto, and any bonding method capable of generating heat by the exothermic reaction without easily removing the bonded metal particles 112 by external force and without hindering contact with the target gas, can be applied.

When the concentration of hydrogen in the air is between 1 to 4%, the composite structure 110 generates heat within the range from 30 to 110° C., preferably, within the range from 39 to 102° C. When the composite structure 110 reacts with hydrogen to generate heat within the range, the generated heat is transferred to the thermochromic layer 200, and the color of the thermochromic particles is changed by the transferred heat.

Thermochromic particles change color according to temperature. As described above, the color change according to temperature of the thermochromic particles may have reversible or irreversible characteristics depending on the components thereof. In this instance, the target temperature can vary depending on the components of the thermochromic particles included in the thermochromic catalyst layer 600.

The thermochromic catalyst layer 600 may include thermochromic particles with reversible characteristics, or thermochromic particles with irreversible characteristics. Alternatively, the thermochromic catalyst layer 600 may include both of thermochromic particles with reversible characteristics, and thermochromic particles with irreversible characteristics.

The thermochromic particles can include dye and developer, and the thermochromic layer 200 with the irreversible characteristics can additionally include wax. The target temperature can vary depending on the components and composition of the thermochromic particles.

The thermochromic particles are the same as those described in the previous examples, so a detailed description will be omitted.

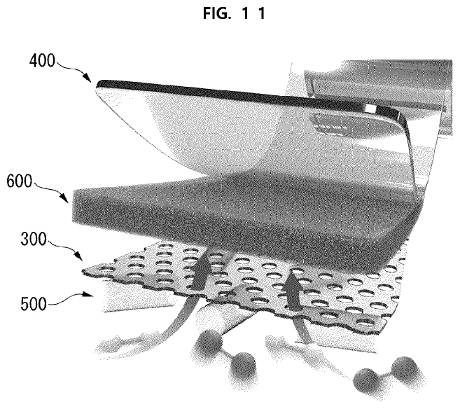

FIG. 11 is a view illustrating a color-changing sensor for gas detection of a combined layer type, which further includes a diffusion space layer type composite structure (layer-by-layer type & combined layer type with spacers).

Referring to FIG. 11, the layer-by-layer type color-changing sensor for gas detection 20 of another embodiment can further include a spacer 500.

Specifically, a spacer layer 500 can be added beneath the adhesive layer 300 to secure the diffusion space for hydrogen and expand the space for oxygen in the air to enter. Accordingly, it allows for a greater amount of target gas to be captured and introduced into the thermochromic catalyst layer 600, thereby increasing the amount of target gas entering the thermochromic catalyst layer 600 and enhancing the heat sensitivity.

Manufacturing Method for the Combined Layer Type Color-Changing Sensor for Gas Detection

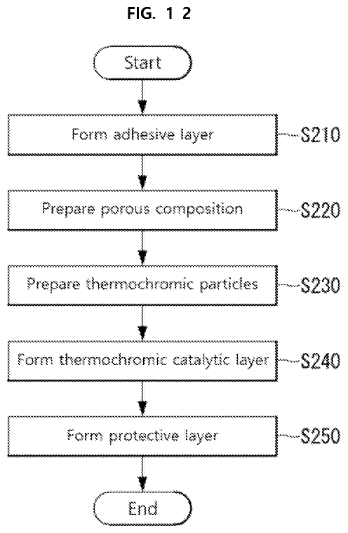

FIG. 12 illustrates a manufacturing method of a color-changing sensor for gas detection according to another embodiment of the present invention.

A porous adhesive layer 300 is formed (S210). In this instance, the formation of adhesive layer 300 is the same as the previous embodiment, so a detailed description will be omitted.

A composite structure is manufactured (S220). In this instance, the manufacturing of the composite structure is the same as the previous embodiment, so a detailed description will be omitted.

Thermochromic particles are produced (S230). In this instance, the manufacturing of the thermochromic particles is the same as the previous embodiment, so a detailed description will be omitted.

The composite structure and thermochromic particles are mixed to form a thermochromic catalyst layer 600 (S240). The composite structure and thermochromic particles can be mixed in equal ratios. The thermochromic catalyst layer 600 can be formed by applying a coating solution in which the composite structure and thermochromic particles are mixed.

Specifically, the composite structure, the thermochromic particles, binder, and solvent are mixed to make a color-changing catalyst coating solution. The color-changing catalyst coating solution can be applied to one side of the adhesive layer 300 to form the thermochromic catalyst layer 600.

Here, the binder can be polyvinyl acetate (PVA), and the solvent can be isopropyl alcohol (IPA).

In an embodiment, 0.1 g of composite structure, 0.1 g of thermochromic particles, 1 g of PVA binder, and 5 g of IPA can be mixed to prepare the color-changing catalyst coating solution. The coating solution can be prepared by mixing 0.1 g of composite structure, 0.1 g of thermochromic particles, 1 g of PVA binder, and 5 g of IPA at 1500 rpm in a revolutionary paste mixer. The prepared coating solution can be applied to the adhesive layer 300 using a bar coating method to form the thermochromic color-changing layer 200.

A protective layer 400 is formed on one side of the thermochromic catalyst layer 600. In this instance, the formation of the protective layer is the same as the previous example, so a detailed description will be omitted.

### Experimental Example 1

Performance Evaluation of Irreversible Color-Changing Sensor (Examples 1 to 3)

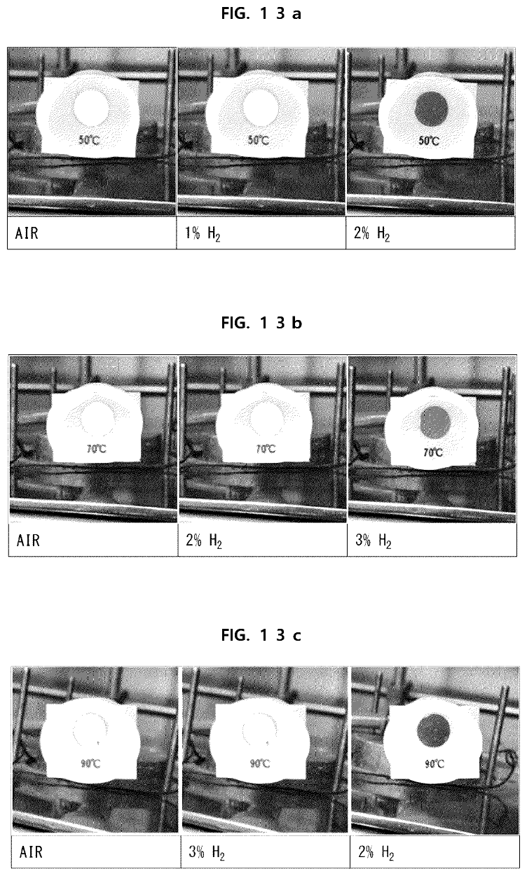

FIGS. 13A to 13C show color change performance evaluation results of an irreversible color-changing sensor which includes a heat color-changing layer 200 by different target temperatures.

Referring to FIG. 13A, a sensor using thermochromic particles (Example 4) at a target temperature of 50° C., when hydrogen concentration is 1% in air, did not show color change at a heating temperature of the catalyst layer 100 of 39.1° C. However, when hydrogen concentration was 2% in air, the sensor caused color change due to the heating temperature reached 52.3° C.

Referring to FIG. 13B, the sensor using thermochromic particles (Example 5) at a target temperature of 70° C., when hydrogen concentration is 2% in air, did not show color change at a heating temperature of the catalyst layer 100 of 52.3° C. However, when hydrogen concentration was 3% in air, the sensor caused color change due to the heating temperature reached 72.7° C.

Referring to FIG. 13C, the sensor using thermochromic particles (Example 6) at a target temperature of 90° C., at room temperature where there is no hydrogen, or when hydrogen concentration is 3% in air, did not show color change at a heating temperature of the catalyst layer 100 of 72.8° C. However, when hydrogen concentration was 4% in air, the sensor caused color change due to the heating temperature reached 101.1° C.

Examples 4 to 6 relate to irreversible color-changing sensors. As described above, the irreversible color-changing sensors maintained the color even when temperature lowered once the color was changed.

### Experimental Example 2

Performance Evaluation of Reversible Color-Changing Sensors (Examples 4 to 6)

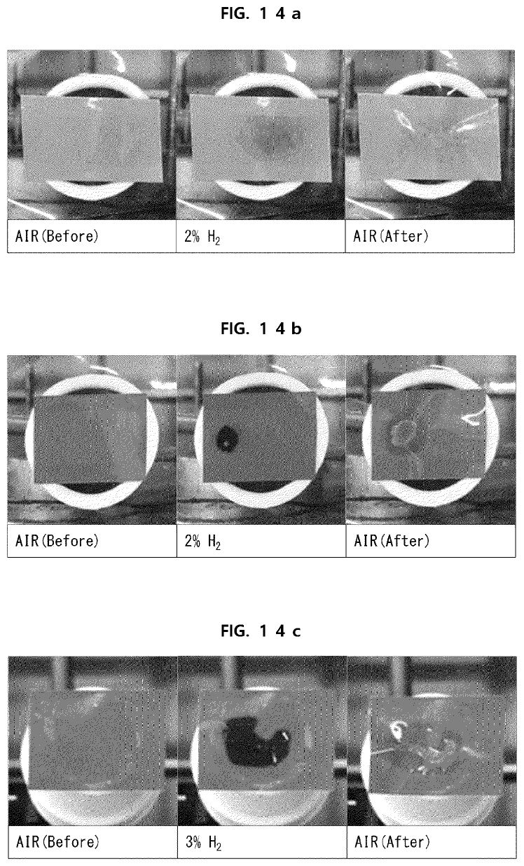

FIGS. 14A to 14C show color change performance evaluation results of a reversible color-changing sensor which includes a heat color-changing layer 200 by different target temperatures.

In case of color-changing sensors with target temperatures of 50° C. (Example 1), 70° C. (Example 2), and 90° C. (Example 3), when heat generation above the target temperature occurred by exposure of hydrogen gas, color change occurred, and then, when the contact with hydrogen was discontinued, the color-changed sensors returned to their original color.

Referring to FIG. 14A, in a case in which the reversible thermochromic particles (Example 1) with a target temperature of 50° C. was used, only air exists, but when the hydrogen gas concentration reached 2%, color change occurred while the heating temperature reached 52.3° C. Thereafter, when hydrogen gas was removed, the temperature dropped below the target temperature of 50° C., and the original color was restored.

As illustrated in FIG. 14B, in a case in which the reversible thermochromic particles (Example 2) with a target temperature of 70° C. was used, only air exists, but when the hydrogen gas concentration reached 3%, color change occurred while the heating temperature reached 72.8° C. Thereafter, when hydrogen gas was removed, the temperature dropped below the target temperature of 72.8° C., and the original color was restored.

In FIG. 14C, in a case in which the reversible thermochromic particles (Example 3) with a target temperature of 90° C. was used, only air exists, but when the hydrogen gas concentration reached 4%, color change occurred while the heating temperature reached 101.1° C. Thereafter, when hydrogen gas was removed, the temperature dropped below the target temperature of 101.1° C., and the original color was restored.

### Experimental Example 3

Performance Evaluation for Use of Joint

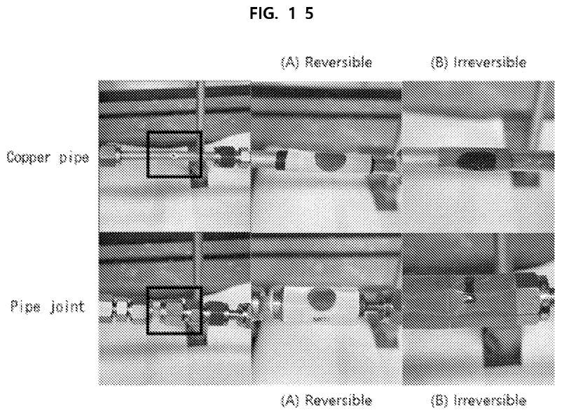

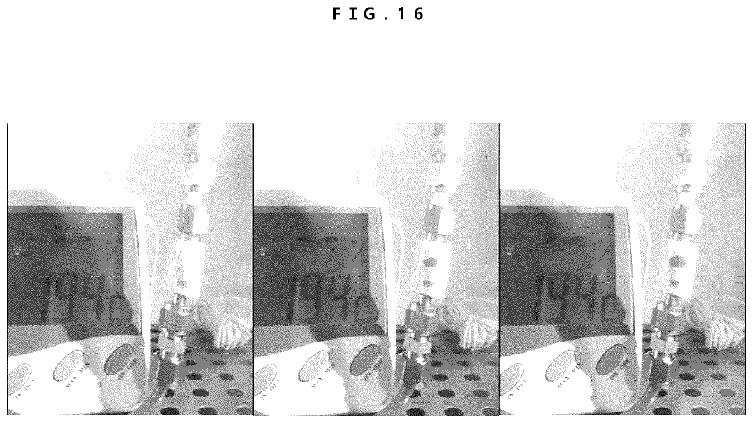

FIG. 15 shows an experiment to detect a hydrogen leak at a joint part between a copper pipe body and a SUS (stainless steel) pipe. The color-changing sensor of the present invention was adhered to the joint part between the copper pipe body and the SUS pipe.

In this case, both the reversible color-changing sensors (Examples 1 to 3) and the irreversible color-changing sensors (Examples 4 to 6) clearly indicated due to the heat generated in the catalytic layer 100 when hydrogen gas is exposed.

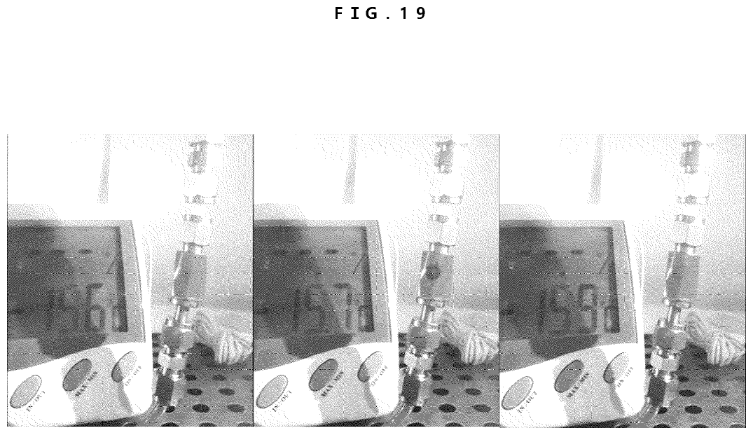

FIGS. 16 to 19 illustrate evaluations of hydrogen gas detection performance at the joint part using the irreversible and reversible color-changing sensors of the present invention. The evaluation was conducted through video observation to evaluate performance under room temperature conditions and sub-zero conditions and monitor changes due to gas exposure and cessation, and images were captured over time.

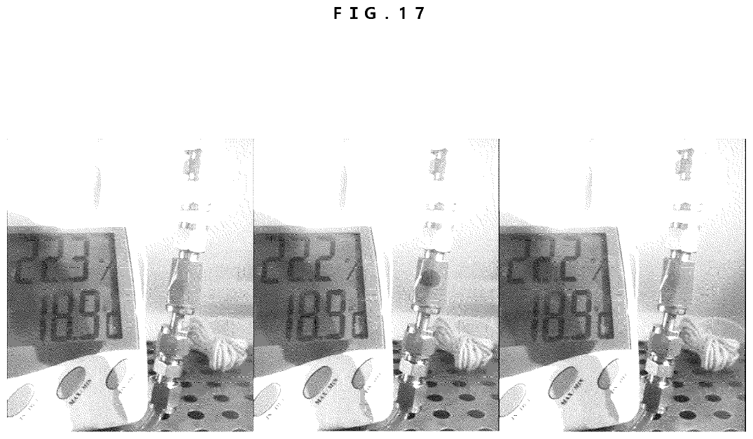

FIGS. 16 and 17 illustrate evaluation of hydrogen detection performance of the irreversible color-changing sensor (FIG. 15) and reversible color-changing sensor (FIG. 16) under room temperature conditions (18.9 to 19.4° C.)

In the examples 3 and 6, the color-changing sensors including the thermochromic layers 200 were used. It was confirmed that both of the irreversible and reversible color-changing sensors caused thermochromic changes in the same humidity range and temperature range as they exceeded the target temperature of 90° C. It was confirmed that the reversible color-changing sensors returned to the original color while hydrogen decreased.

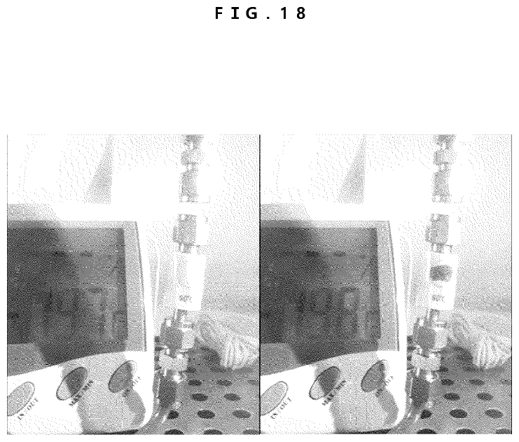

FIGS. 17 and 18 illustrate evaluation of the detection of hydrogen under sub-zero conditions. It was confirmed that both of the irreversible and reversible color-changing sensors caused color change due to heat generation under the sub-zero conditions as they exceeded the target temperature of 90° C. It was confirmed that the reversible color-changing sensors returned to the original color while hydrogen decreased.

Although the present invention has been described in detail with reference to preferred embodiments, the scope of the invention is not limited thereto. Various modifications and changes made by those skilled in the art using the basic concept of the invention defined in the following claims also fall within the scope of the invention.

The present invention relates to a color-changing sensor for gas detection and a method for manufacturing the same. The color-changing sensor for gas detection can be used in various ways since easily verifying the presence of hydrogen gas through color-change when contacting with hydrogen gas, and show reversible characteristics to return to its original color in case of non-contact with hydrogen gas or irreversible characteristics to maintain the changed color. Furthermore, the color-changing sensor is applicable to various fields since being used in various types, such as a tape type or a spray type, is convenient to use as detecting hydrogen in the air without the need for electrical power, and can be used not only at room temperature but also at sub-zero temperature.

1. A color-changing sensor for gas detection comprising:
an adhesive layer;
a catalytic layer; and
a thermochromic layer,
wherein the catalytic layer includes a porous support bonded to metal particles.

2. The color-changing sensor according to claim 1, wherein the metal particles are selected from a group consisting of precious metal catalysts, precious metal alloy catalysts, non-precious metal catalysts, non-precious metal alloy catalysts, metal oxides, metal chlorides, metal complexes, and mixtures thereof.

3. The color-changing sensor according to claim 1, wherein the porous support is selected from the group consisting of graphene, carbon allotropes, ceramic oxides, and mixtures thereof.

4. The color-changing sensor according to claim 1, wherein the thermochromic layer includes thermochromic particles, and
wherein the thermochromic particles include dye and developer.

5. The color-changing sensor according to claim 4, wherein the thermochromic layer includes a thermochromic coating composition, and
wherein the thermochromic coating composition comprises thermochromic particles, a binder, and a solvent.

6. The color-changing sensor according to claim 1, wherein the adhesive layer has a porous structure.

7. The color-changing sensor according to claim 1, further comprising:
a spacer.

8. The color-changing sensor according to claim 1, wherein the gas is selected from the group consisting of hydrogen gas, methane gas, ethane gas, propane gas, butane gas, and mixtures thereof.

9. The color-changing sensor according to claim 1, wherein the catalytic layer generates heat at temperature between 30 and 110° C. when the concentration of hydrogen in the air is between 1 and 4%.

10. The color-changing sensor according to claim 1, wherein the color-changing sensor detects exposure of the target gas under conditions ranging from −20 to 20° C.

11. (canceled)

12. A method for manufacturing a color-changing sensor for gas detection, comprising the steps of:
manufacturing a porous support, on the surface of which metal particles are bonded;
bonding the porous support to one side of an adhesive layer; and
preparing a thermochromic coating composition by dissolving thermochromic particles and a binder in a solvent and coating thermochromic coating composition on one side of the porous support, on the surface of which metal particles are bonded, to form a thermochromic layer.

13. The method according to claim 12, wherein the metal particles are selected from a group consisting of precious metal catalysts, precious metal alloy catalysts, non-precious metal catalysts, non-precious metal alloy catalysts, metal oxides, metal chlorides, metal complexes, and mixtures thereof.

14. The method according to claim 12, wherein the porous support is selected from the group consisting of graphene, carbon allotropes, ceramic oxides, and mixtures thereof.

15. The method according to claim 12, wherein the thermochromic particles include dye and developer.

16-19. (canceled)
